(19) United States
(12) Patent Application Publication
(10) Pub. No.: US 2016/0274303 A1
(43) Pub. Date: Sep. 22, 2016

(54) OPTICAL FIBER

(71) Applicant: TERUMO KABUSHIKI KAISHA, Tokyo (JP)

(72) Inventors: Yuuichi TADA, Tokyo (JP); Yuuki ITOU, Hadano-city (JP); Kazuaki KANAMOTO, Hadano-city (JP)

(73) Assignee: TERUMO KABUSHIKI KAISHA, Tokyo (JP)

(21) Appl. No.: 15/074,815

(22) Filed: Mar. 18, 2016

(30) Foreign Application Priority Data
Mar. 19, 2015 (JP) 2015-055638

(51) Int. Cl. G02B 6/26; A61N 5/06; F21V 8/00

(52) U.S. Cl. CPC G02B 6/262; G02B 6/001; A61N 5/0601; A61N 2005/063; A61N 2005/0602

(57) ABSTRACT

An optical fiber is capable of radiating light radially in a wide range. The optical fiber includes an elongate primary coated optical fiber adapted to transmit light radiated for medical treatment. The primary coated optical fiber includes a core at a central portion and a cladding covering the core, and has an irradiation groove along the circumferential direction thereof. The irradiation groove extends from the cladding to reach the core of the primary coated optical fiber, and has side portions on both sides with respect to the axial direction of the primary coated optical fiber and a bottom portion. The side portions each have a protruding portion shaped to be protrude toward the inside of the irradiation groove.

### CROSS-REFERENCES TO RELATED APPLICATIONS

This application claims priority to Japanese Application No. 2015-055638 filed on Mar. 19, 2015, the entire content of which is incorporated herein by reference.

### TECHNICAL FIELD

The present disclosure relates to an optical fiber, particularly to an optical fiber by which light transmitted in the axial direction is radiated in radial directions from an irradiation groove formed along the circumferential direction of a core.

### BACKGROUND DISCUSSION

A known therapy involves irradiation of a lesion in a blood vessel with light of a predetermined wavelength that is emitted from an optical fiber. One example of such a therapy is photodynamic therapy (PDT), in which a photosensitive substance is injected into a living body, and a biological tissue as a target is irradiated with light of a predetermined wavelength, in order to generate active oxygen from the photosensitive substance and treat the lesion with the active oxygen.

An optical fiber to be used for such an application should have a structure in which light transmitted in the axial direction is radiated in radial directions from a predetermined position. One such structure includes a primary coated optical fiber formed with an irradiation groove V-shaped in section and extending in the circumferential direction, such that light is radiated in radial directions from the irradiation groove. An example of an optical fiber having such a structure is described in U.S. Patent Application Publication No. 2009/0240242.

### SUMMARY

The sectional shape of the irradiation groove in an optical fiber according to the related art is a V shape in which rectilinear side portions on both sides of the groove are joined at an acute angle at a bottom portion of the groove. With the irradiation groove being formed in such a shape that is composed of rectilinear portions, the resultant range of irradiation with light is narrow. It is, however, desirable that radiation of light toward a target be performed uniformly and in a wide range. With the shape of the irradiation groove in the optical fiber according to the related art, however, the matter to be irradiated is irradiated locally with light of high energy density. In order to solve the above-described problem, an object of the present disclosure is to provide an optical fiber by which light can be radiated radially in a wide range.

In one aspect of the present disclosure, there is provided an optical fiber including an elongate primary coated optical fiber adapted to transmit light radiated for medical treatment, wherein the primary coated optical fiber includes a core at a central portion and a cladding covering the core, and has an irradiation groove along a circumferential direction thereof, the irradiation groove extends from the cladding to reach the core of the primary coated optical fiber, and has side portions on both sides with respect to an axial direction of the primary coated optical fiber and a bottom portion, and the side portions each have a protruding portion shaped to protrude toward an inside of the irradiation groove.

In the optical fiber configured as above, light entering the optical fiber in the axial direction of the core is emitted in a wide angular range in radial direction by the protruding portions. Accordingly, light can be radiated in a wide range from the irradiation groove.

### BRIEF DESCRIPTION OF THE DRAWINGS

FIG. 1 is a perspective view showing a vicinity of a distal of an optical fiber according to an embodiment of the present disclosure;

FIG. 2 is a sectional view showing a vicinity of the distal of the optical fiber;

FIG. 3 is a figure illustrating a sectional shape of an irradiation groove;

FIGS. 4A and 4B are figures illustrating a sectional shape of an irradiation groove formed in a V shape and the sectional shape of the irradiation groove in the present embodiment, in contrast;

FIG. 5 is a front view of a primary coated optical fiber in a vicinity of the irradiation groove;

FIG. 6 is a front view of a primary coated optical fiber having an irradiation groove according to a first modification;

FIG. 7 is a front view of a primary coated optical fiber having an irradiation groove according to a second modification;

FIG. 8 is a figure illustrating a sectional shape of an irradiation groove according to a third modification;

FIG. 9 is a figure showing a layout of irradiation grooves in a primary coated optical fiber according to a fourth modification; and

FIG. 10 is a figure showing a layout of irradiation grooves in a primary coated optical fiber according to a fifth modification.

### DETAILED DESCRIPTION

One embodiment of the present disclosure will be described below, referring to the drawings. Note that dimensional ratios in the drawings may be exaggerated for convenience of explanation and may therefore be different from the actual ratios. In addition, herein the side toward which an optical fiber 10 is inserted into a body lumen will be referred to as “distal” or “distal end,” and the side of the operator's proximal operation will be referred to as “proximal” or “proximal end.”

The optical fiber 10 according to one embodiment of the present disclosure is inserted into a body lumen such as a blood vessel, to be used to irradiate an affected part with light for the purpose of treating the affected part. The light with which to irradiate the affected part is light ordinarily used for intraluminal treatment, and is selected from lights with wavelengths of 810 nm, 980 nm, 1,470 nm, and 2,000 nm, for example. Note that light with other wavelengths may also be used. As a light source for the light to be radiated into the optical fiber 10, a laser device (not shown) or the like is used.

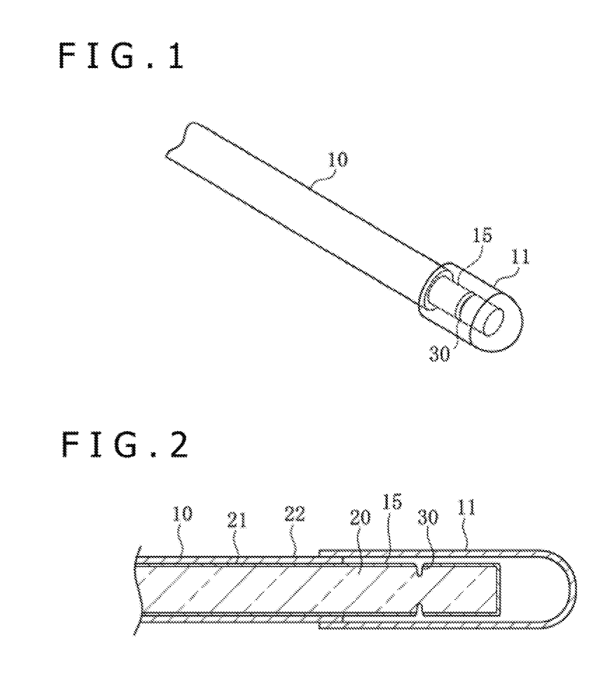

As shown in FIG. 1, a cap 11 is provided at a distal portion of the optical fiber 10. At the distal portion of the optical fiber 10 covering the cap 11, a protective layer of the optical fiber 10 has been removed, to expose a primary coated optical fiber 15. At this part, there is formed an irradiation groove 30 through which light transmitted in the axial direction of the optical fiber 10 is emitted in the direction of the circumference.

As shown in FIG. 2, the optical fiber 10 has the primary coated optical fiber 15 including a cladding 21 provided around an outer periphery of a fiber-shaped core 20 so as to cover the core 20, and, further, a protective layer 22 is provided around an outer periphery of the cladding 21. The core 20 is formed from a dielectric material such as silica glass, multi-component glass, or plastic. The cladding 21 is formed from a dielectric material such as silica glass, multi-component glass, silicone, fluoropolymer, or plastic. The core 20 is hollow cylindrical in shape, and the cladding 21 is concentric with the core 20. The protective layer 22 is formed from a resin. It is to be noted, however, that the optical fiber 10 may be formed from other material. In this figure and subsequent figures, the left side in the drawing is the proximal end, and the right side in the drawing is the distal end. The core 20 is higher than the cladding 21 in refractive index. This ensures that light undergoes total reflection at a boundary surface of the cladding 21, so that the light is transmitted in the state of being enclosed in the core 20.

The cap 11 is formed from a light-transmitting material such as glass. In addition, a space between the cap 11 and the primary coated optical fiber 15 of the optical fiber 10 constitutes an air layer. This ensures that light having been transmitted in the axial direction of the core 20 of the optical fiber 10 undergoes refraction and reflection at the irradiation groove 30, thereby changes in propagation direction toward the circumference, and is emitted to the exterior through the cap 11. Note that other than the air layer, a material with a different refractive index such as nitrogen or rare gas can be used to fill the space between the cap 11 and the primary coated optical fiber 15 of the optical fiber 10, or a fluid such as silicone oil can be disposed in the space.

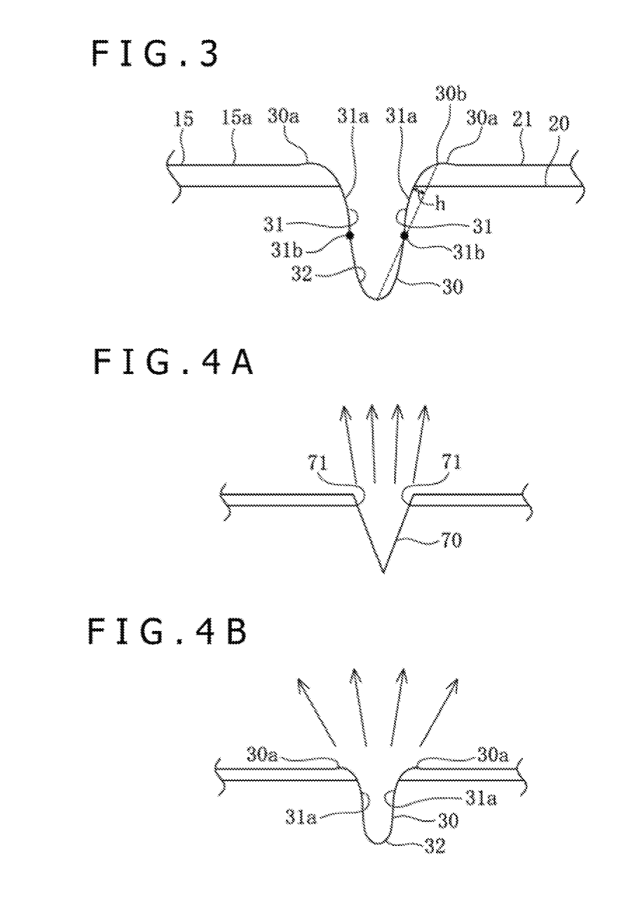

The shape of the irradiation groove 30 will be described in detail. The irradiation groove 30 is formed along the whole circumference of the primary coated optical fiber 15 in such a manner as to penetrate the cladding 21 to reach the core 20. As shown in FIG. 3, the sectional shape of the irradiation groove 30 is defined by a continuous curve not having any rectilinear portion or any angular portion. The irradiation groove 30 is formed with side portions 31 on both sides opposed in the axial direction of the primary coated optical fiber 15 and with a bottom portion 32 of the groove, the side portions 31 and the bottom portion 32 being formed in a mutually continuous shape.

At edge portions on both sides which form an opening (entrance) of the irradiation groove 30, there are formed edge protuberant portions 30a which are protuberant outward from an outer peripheral surface 15a of the primary coated optical fiber 15. Herein the highest positions of the edge protuberant portions 30a are deemed as edge portions 30b of the irradiation groove 30. The side portions 31 are each formed in a curved shape from the edge portion 30b toward the bottom portion 32 of the irradiation groove 30. The side portion 31 has a protruding portion 31 a so shaped as to protrude toward the inside of the irradiation groove 30, with reference to a straight line (indicated by broken line in the figure) connecting the edge portion 30b and the deepest point of the bottom portion 32 of the irradiation groove 30. The protruding portion 31a protrudes by a maximum depth h indicated in the figure from the straight line connecting the edge portion 30b and the deepest point of the bottom portion 32 of the irradiation groove 30. In addition, the protruding portion 31 a interconnects continuously from the edge protuberant portion 30a to the bottom portion 32.

The irradiation groove 30 has inflection points 31b at lower portions of the protruding portions 31a. On the upper side of the inflection point 31b of the irradiation groove 30, a center position defining a radius of curvature of the upper-side portion lies on the inside of the surface of the irradiation groove 30. On the other hand, on the lower side of the inflection point 31b of the irradiation groove 30, a center position defining a radius of curvature of the lower-side portion lies on the outside of the surface of the irradiation groove 30. The inflection points 31b therefore define a transition between a convex upper-side portion and a concave lower-side portion. The portions upwardly of the inflection points 31b of the irradiation groove 30 are made to be the side portions 31, and the portion downwardly of the inflection points 31b of the irradiation groove 30 is made to be the bottom portion 32. The bottom portion 32 of the irradiation groove 30 is formed in a curved shape continuously interconnecting the side portions 31 on both sides; specifically, the bottom portion 32 is U-shaped in section.

The irradiation groove 30 thus shaped can radiate light to a wide range as compared with a V-shaped groove that has rectilinear portions and an angular portion in section. In FIGS. 4A and 4B, light emission directions are schematically indicated. FIG. 4A shows a sectional shape of an irradiation groove 70 formed in a V shape. The irradiation groove 70 is formed in a sectional shape such that two rectilinear side portions 71 are joined at a bottom portion. In this case, rays of light undergo refraction and reflection in substantially the same direction, so that the light is emitted in the radial direction of the core 20, with little spreading.

On the other hand, FIG. 4B shows the shape of the irradiation groove 30 in the present embodiment. In this case, since the side portions 31 have the protruding portions 31a, rays of light undergo refraction and reflection at different angles depending on the positions the rays reach, so that the light is emitted at a wider angle. In addition, the edge portions of the irradiation groove 30 form the edge protuberant portions 30a, which also produces an effect of spreading the emission angle of light. Further, the bottom portion 32 is formed in a curved shape, which also produces an effect of spreading the light emission angle.

These effects ensure that the light from the irradiation groove 30 is radiated to a wide range, as compared with the V-shaped irradiation groove 70. As a result, a wide range of a matter to be irradiated can be irradiated with light at one time, which contributes to shortening of treatment time. Besides, a peak value of energy density of the light radiated can be reduced, so that uniform irradiation with light can be performed and, therefore, medical treatment can be facilitated.

The irradiation groove 30 is formed by irradiating the primary coated optical fiber 15 with laser light. In forming the irradiation groove 30, the protective layer 22 at a distal portion of the optical fiber 10 is removed in advance. Next, the optical fiber 10 is fixed to a manufacturing apparatus. While the optical fiber 10 is rotated about its axis, an outer peripheral surface of the optical fiber 10 is irradiated with laser light, so as to cause melting or transpiration of the cladding 21 and surface of the core 20 of the primary coated optical fiber 15, thereby forming the irradiation groove 30. When the irradiation groove 30 is thus formed by irradiation with laser light, the curved surface shape can be easily formed.

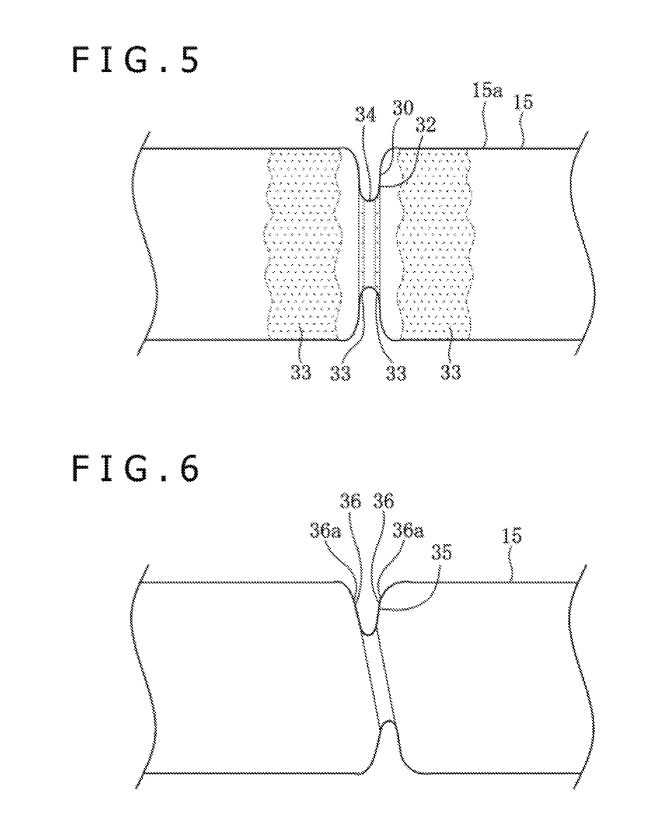

As shown in FIG. 5, rough portions 33 having rough surfaces are formed at those portions of the outer peripheral surface 15a of the primary coated optical fiber 15 which are at both peripheral ends of the irradiation groove 30 on both sides of the irradiation groove 30, and in the periphery of a deepest portion 34 of the irradiation groove 30. In other words, no rough portion 33 is present at the deepest portion 34, and a region of the rough portion 33 and a region free of the rough portion 33 are alternately formed as the distance from the deepest portion 34 increases in the axial direction. Each of the rough portions 33 is formed by minute projections and recesses in the surface of the optical fiber 10, the size of the projections and recesses being at least greater than the wavelength of the light transmitted by the optical fiber 10. Therefore, part of the light transmitted in the axial direction of the optical fiber 10 undergoes irregular reflection, to be emitted in the radial directions of the optical fiber 10. Since the rays of light emitted from the rough portions 33 are oriented in various directions due to the irregular reflection, these rays can constitute part of irradiation light covering a wide range, together with the rays of light emitted from the irradiation groove 30. While the rough portion 33 is absent at the deepest portion 34 of the irradiation groove 30 and the rough portions 33 are formed in the periphery of the deepest portion 34 in FIG. 5, the rough portion 33 may be formed at the deepest portion 34.

The rough portions 33 are formed, for example, by spattering or scattering of molten material of the primary coated optical fiber 15 in the process of forming the irradiation groove 30 by irradiation with laser light. Note that the method for forming the rough portions 33 is not restricted to this process. For example, the rough portions 33 may be formed by subjecting the primary coated optical fiber 15 to surface roughening after the formation of the irradiation groove 30.

A first modification of the irradiation groove will now be described. As shown in FIG. 6, an irradiation groove 35 in this modification is formed to be inclined against a plane orthogonal to the axial direction of the primary coated optical fiber 15. Therefore, side portions 36 on both sides are individually varied in shape along the circumferential direction, but both of them are so shaped as to have protruding portions 36a. Such shapes can be formed by inclining the primary coated optical fiber 15 with reference to the angle of incidence of laser light at the time of forming the irradiation groove 35.

Where the irradiation groove 35 is thus formed at an inclination against the plane orthogonal to the axial direction of the primary coated optical fiber 15, the width of the irradiation groove 35 in the axial direction can be enlarged. As a result, light can be radiated in a wider range.

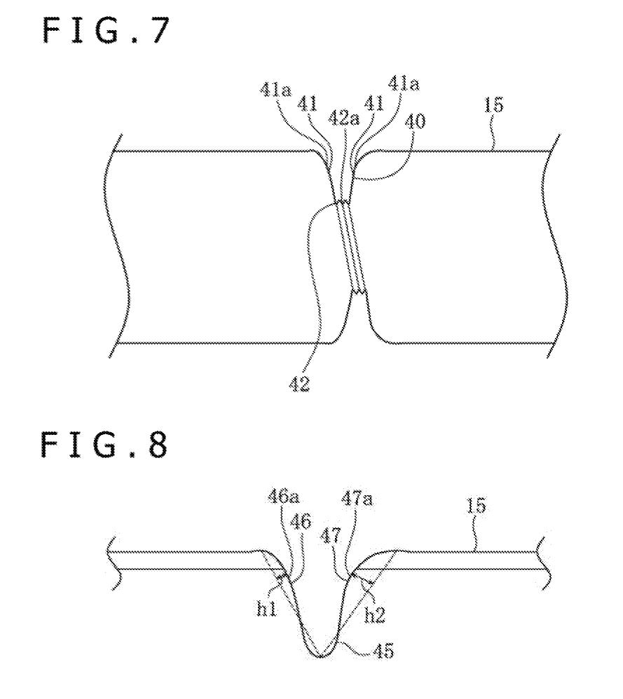

A second modification of the irradiation groove will be described below. As shown in FIG. 7, an irradiation groove 40 in this modification has protruding portions 41a at side portions 41, like the above-mentioned irradiation grooves. However, the irradiation groove 40 is formed with a spiral groove 42a along the circumferential direction, at a bottom portion 42 thereof. The groove 42a is formed at the deepest portion of the bottom portion 42. Such a shape can be formed by softening a core 20 by heating and twisting the softened core 20, at the time of forming the irradiation groove 40.

When the groove 42a is thus formed at the bottom portion 42 of the irradiation groove 40, more light can be emitted in the radial direction at the bottom portion 42. In addition, since the groove 42a is spiral in shape, a plurality of projections and recesses are formed in the axial direction of the bottom portion 42, so that light can be emitted in a greater width. As a result, together with the light emitted from the side portions 41 of the irradiation groove 40, the quantity of light radiated in the radial direction can be increased, and the light can be emitted in a broader width. Note that while the groove 42a is spirally shaped in this modification, a plurality of grooves may be formed at positions spaced apart along the axial direction.

A third modification of the irradiation groove will now be described. As shown in FIG. 8, an irradiation groove 45 in this modification has a structure in which a side portion 46 on one side and a side portion 47 on the other side are different in shape. The side portion 46 on one side has a protruding portion 46a with a maximum protruding depth of h1, while the side portion 47 on the other side has a protruding portion 47a with a maximum protruding depth of h2, wherein h1<h2. Thus, the protruding portion 47a of the side portion 47 is greater in the extent of protrusion. In this case, the radius of curvature of the protruding portion 46a on one side is smaller than that of the protruding portion 47a on the other side.

Where the irradiation groove 45 thus has the side portions 46 and 47 formed by curved surfaces with different radii of curvature, the energy density of light on the proximal end of the irradiation groove 45 and that on the distal end of the irradiation groove 45 can be made to be different. In this modification, the side portion 47 on the distal end has a greater radius of curvature, so that light is emitted therefrom to a wider range. On the other hand, the side portion 46 on the proximal end has a smaller radius of curvature, so that light is emitted therefrom to a narrower range. As a result, the energy density of light radiated is lower on the distal end of the irradiation groove 45, while the energy density of light radiated is higher on the proximal end of the irradiation groove 45. Irradiation light thus different in energy density of light between the distal end and the proximal end can be used, as required, according to conditions of the matter to be irradiated with light or the like factors. In addition, the radius of curvature of the side portion 46 on the proximal end may be set larger and that of the side portion 47 on the distal end may be set smaller, conversely to this modification.

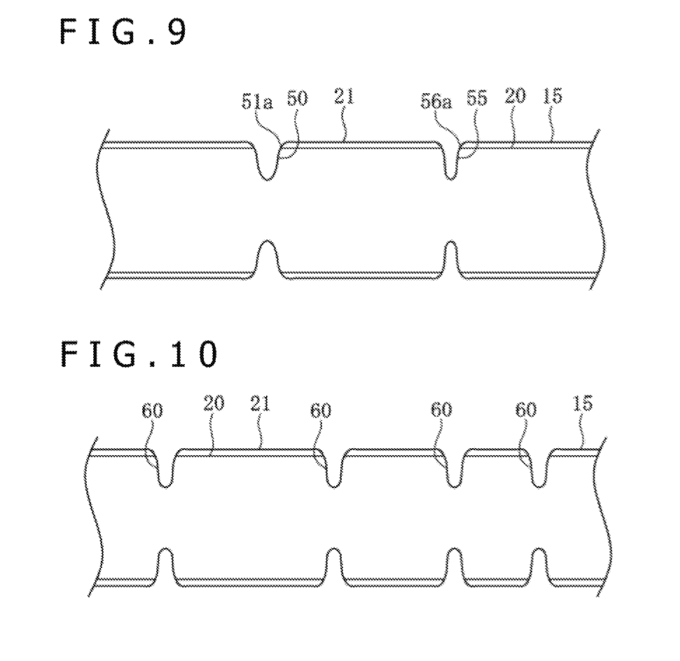

A fourth modification of the irradiation groove will now be described. In each case of the irradiation groove as above, one irradiation groove has been provided in the primary coated optical fiber 15. However, a plurality of irradiation grooves may be formed at positions spaced apart along the axial direction of the primary coated optical fiber 15. As shown in FIG. 9, in this modification, two irradiation grooves 50 and 55 are formed at positions spaced apart along the axial direction of a primary coated optical fiber 15. The irradiation groove 55 disposed on the distal end is formed to be smaller in groove width than the irradiation groove 50 disposed on the proximal end. Note that the irradiation grooves 50 and 55 are equal in groove depth. In addition, a cladding 21 is disposed between the irradiation grooves 50 and 55.

The irradiation grooves 50 and 55 can emit light to a wider range as the groove width is greater. In addition, the irradiation grooves 50 and 55 can emit light to a wider range as the radius of curvature of protruding portions 51a and 56a are greater. Besides, the irradiation grooves 50 and 55 can emit more light in the radial direction as each of the groove depth is larger. Therefore, the irradiation groove 50 on the proximal end that is greater in groove width can emit light to a wide range, as compared with the irradiation groove 55 on the distal end. On the other hand, the irradiation groove 55 on the distal end that is smaller in groove width emits light to a narrower range, and the energy density of the light emitted is higher accordingly.

In the case where the primary coated optical fiber 15 is provided with a plurality of irradiation grooves 50 and 55, part of the light entering in the axial direction of the primary coated optical fiber 15 is emitted by the irradiation groove 50 on the proximal end, so that the intensity of light at the position of the irradiation groove 55 on the distal end has been attenuated accordingly. In this modification, the irradiation groove 55 is set smaller than the irradiation groove 50 in groove width, and the groove width is so adjusted that the energy density of light emitted at the irradiation groove 55 will be equivalent to that at the irradiation groove 50 on the proximal end. By this adjustment, light can be radiated uniformly to a wider range.

Thus, in this modification, the irradiation grooves 50 and 55 that are different in groove width are provided. In the cases where a plurality of irradiation grooves are provided, it is possible, by adjusting the groove width or groove depth of each irradiation groove or the radius of curvature of the protruding portion of each irradiation groove, to obtain irradiation light uniformly in a wider range in the axial direction, or to obtain irradiation light at an intensity varying along the axial direction.

A fifth modification of the irradiation groove will be described below. In this modification, also, a plurality of irradiation grooves 60 are arranged along the axial direction, as shown in FIG. 10. These irradiation grooves 60 are formed in the same shape. On the other hand, the interval of the irradiation grooves 60 is larger on the proximal end and smaller on the distal end.

As has been described above, the light entering from the proximal end is emitted in the radial direction at each irradiation groove 60 with corresponding attenuation, so that the intensity of light is weakened along the distal direction. In this modification, the interval of the irradiation grooves 60 is regulated to be smaller on the more distal end. This ensures that, on the distal end where the light intensity is low, rays of light emitted at adjacent irradiation grooves 60 overlap with each other. Consequently, irradiation light can be obtained uniformly in a wide range along the axial direction.

Note that in the cases where the interval of the irradiation grooves 60 varies, the irradiation grooves 60 can be so arranged as to obtain light uniformly along the axial direction, as in this modification, and can be so arranged as to obtain light at an intensity that varies along the axial direction.

As has been described above, the optical fiber 10 according to the present embodiment includes the elongate primary coated optical fiber 15 adapted to transmit light radiated for medical treatment. The primary coated optical fiber 15 includes the core at a central portion and the cladding covering the core, and has the irradiation groove 30 along the circumferential direction thereof. The irradiation groove 30 extends from the cladding to reach the core of the primary coated optical fiber 15, and has the side portions 31 on both side in the axial direction of the primary coated optical fiber 15 and the bottom portion 32. Each of the side portions 31 has the protruding portion 31a shaped to protrude toward the inside of the irradiation groove 30. By this structure, light entering in the axial direction of the core 20 is emitted by the protruding portion 31a to a wide angular range, so that light can be radiated to a wide range from the irradiation groove 30.

Where the bottom portion 32 is formed in a curved shape such as to continuously interconnect the side portions 31 on both sides, the light emitted from the bottom portion 32 can also have a wide angular range.

Where the irradiation groove 30 is formed at edge portions thereof with the edge protuberant portions 30a which are protuberant as compared to the peripheral surface of the primary coated optical fiber 15, the light emission angle can be widened by the edge protuberant portions 30a.

Where the irradiation groove 30 have the inflection points 31b at lower portions of the protruding portions 31a in the shape in a section orthogonal to the axial direction of the primary coated optical fiber 15, the bottom portion 32 can be curved in a U shape, while making the opening side of the irradiation groove 30 as the protuberant portion 31a. As a result, the irradiation groove 30 can be shaped for radiating light to a wide range.

Where the irradiation groove 35 is formed to be inclined against a plane orthogonal to the axial direction of the primary coated optical fiber 15, the width of the irradiation groove 35 in the axial direction can be enlarged, whereby light can be radiated to a wider range.

Where the bottom portion 42 is provided with a plurality of grooves or spiral groove 42a along the circumferential direction, a larger amount of light can be emitted at the bottom portion 42a, and light can be radiated to a wider range.

Where a plurality of irradiation grooves 50 and 55 are formed at positions spaced apart along the axial direction of the primary coated optical fiber 15 and the cladding 21 is disposed between the irradiation grooves 50 and 55, light can be radiated to a wider range. In addition, by varying parameters such as the groove width and the pitch of the irradiation grooves, light radiation conditions can be controlled variously.

Where the rough portions 33 including a surface having projections and recesses greater than the wavelength of the light to be radiated are formed in the range of either of the irradiation groove 30 and a region including the peripheral portion of the irradiation groove 30, it is possible to emit light having undergone irregular reflection at the rough portions 33, and thereby to radiate light to a wider range.

Note that the present invention is not restricted only to the aforementioned embodiment, and various modifications can be made by those skilled in the art, within the scope of technical thought of the invention. For instance, in the aforementioned embodiment, the distal surface of the primary coated optical fiber 15 is composed of the cladding 21 as shown in FIG. 2, so that light is not emitted from the distal surface. However, the cladding at the distal surface of the primary coated optical fiber 15 may be partly cut out to permit light to be radiated also through the distal surface.

It should be understood by those skilled in the art that various modifications, combinations, sub-combinations and alterations may occur depending on design requirements and other factors insofar as they are within the scope of the appended claims or the equivalents thereof.

The detailed description above describes an optical fiber. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An optical fiber comprising:
an elongate primary coated optical fiber adapted to transmit light radiated for medical treatment,
wherein the primary coated optical fiber includes a core at a central portion and a cladding covering the core, and has an irradiation groove along a circumferential direction thereof,
the irradiation groove extends from the cladding to reach the core of the primary coated optical fiber, and has side portions on both sides with respect to an axial direction of the primary coated optical fiber and a bottom portion, and
the side portions each have a protruding portion shaped to protrude toward an inside of the irradiation groove.

2. The optical fiber according to claim 1,
wherein the bottom portion is formed in a curved shape such as to continuously interconnect the side portions on both sides.

3. The optical fiber according to claim 1,
wherein an edge protuberant portion protuberant with reference to a peripheral surface of the primary coated optical fiber is formed at an edge portion of the irradiation groove.

4. The optical fiber according to claim 1,
wherein the irradiation groove has an inflection point at a lower portion of the protruding portion, in its shape in a section orthogonal to the axial direction of the primary coated optical fiber.

5. The optical fiber according to claim 1,
wherein the irradiation groove is formed to be inclined against a plane orthogonal to the axial direction of the primary coated optical fiber.

6. The optical fiber according to claim 1,
wherein the bottom portion has a plurality of grooves or a spiral groove along the circumferential direction.

7. The optical fiber according to claim 1,
wherein the side portions on both sides with respect to the axial direction have the same shape.

8. The optical fiber according to claim 1,
wherein the side portions on both sides with respect to the axial direction have different shapes.

9. The optical fiber according to claim 1,
wherein a plurality of the irradiation grooves are formed at positions spaced apart along the axial direction of the primary coated optical fiber, and the cladding is disposed between the irradiation grooves.

10. The optical fiber according to claim 1,
wherein at least two of the plurality of the irradiation grooves have different widths.

11. The optical fiber according to claim 1,
wherein a rough portion including a surface having projections and recesses greater than a wavelength of the light radiated is formed in a range of either of the irradiation groove and a region including a peripheral portion of the irradiation groove.
